(19) United States
(12) Patent Application Publication
(10) Pub. No.: US 2006/0089724 A1
(43) Pub. Date: Apr. 27, 2006

(54) High strength osteoarticular allograft and a method of making the same

(76) Inventor: Richard M. Terek, Providence, RI (US)

Correspondence Address: HAMMER & HANF, PC, 3125 SPRINGBANK LANE, SUITE G, CHARLOTTE, NC, 28226, US

(21) Appl. No.: 11/256,234

(22) Filed: Oct. 21, 2005

Related U.S. Application Data
(60) Provisional application No. 60/620,740, filed on Oct. 21, 2004.

(51) Int. Cl. A61F 2/28

(52) U.S. Cl. 623/23.62; 623/23.63

(57) ABSTRACT

The instant invention is a high strength osteoarticular allograft and a method of making the same. The high strength osteoarticular allograft of the instant invention includes an osteoarticular allograft having an intramedullary canal and a cancellous bone region, and a filler impregnating the intramedullary canal and a portion of the cancellous bone region. The method of making a high strength osteoarticular allograft includes the following steps: (1) providing an osteoarticular allograft having an intramedullary canal and a cancellous bone region; (2) providing a filler; and (3) impregnating the intramedullary canal and a portion of said cancellous bone region with said filler.

### CROSS-REFERENCE TO RELATED APPLICATIONS

This application is a non-provisional application claiming priority from provisional application Ser. No. 60/620,740, filed on Oct. 21, 2004, entitled “AN IMPROVED OSTEOARTICULAR ALLOGRAFT AND METHOD OF MAKING SAME,” the teachings of which are incorporated by reference herein as if reproduced in full below.

### FIELD OF INVENTION

The instant application relates to a high strength osteoarticular allograft and a method of making the same.

### BACKGROUND OF THE INVENTION

Bone is one of the hardest structures of human body, possessing both a certain degree of toughness and elasticity. Bone is composed of two kinds of tissue, one of which is dense in texture known as compact tissue, and the other consist of slender fibers and lamellae which join to form a reticular structure, resembling a lattice-work, known as cancellous tissue. The compact tissue, which is extremely porous, is placed on the exterior of the bone while the cancellous tissue is placed in the interior. Bone, during life, is permeated by vessels, and is enclosed, except where it is coated with auricular cartilage, in a fibrous material, the periosteum, by means of which these vessels reach the hard tissue. The interior of each the long bones of the limbs presents a cylindrical cavity filed with marrow and lined with a highly vascular areolar structure known as medullary membrane.

It is generally known that the reconstruction after interaarticular resection for bone tumor can be performed with osteoarticular allografts, endoprostheses, or allograft/endoprosthetic composites. However, osteoarticular allografts are often the preferred reconstructive option after bone tumor resection.

In general, osteoarticular allografts have their greatest application in reconstruction of knee joints, either proximal tibia or distal femur. Osteoarticular allografts, generally, preserve the opposing joint surfaces, which is particularly advantageous in a skeletally immature patient since the opposing growth plates can be preserved. Osteoarticular allografts also provide a site for reattachment of ligaments for soft tissue reconstruction.

However, there are some primary complications associated with the use of osteoarticular allografts in reconstruction after interaarticular resection for bone tumor. These complications include fracture, non-union, infection, and joint instability.

Therefore, there is a need for a high strength osteoarticular allograft and a method of making the same.

### SUMMARY OF THE INVENTION

The instant invention is a high strength osteoarticular allograft and a method of making the same. The high strength osteoarticular allograft of the instant invention includes an osteoarticular allograft having an intramedullary canal and a cancellous bone region, and a filler impregnating the intramedullary canal and a portion of the cancellous bone region. The method of making a high strength osteoarticular allograft includes the following steps: (1) providing an osteoarticular allograft having an intramedullary canal and a cancellous bone region; (2) providing a filler; and (3) impregnating the intramedullary canal and a portion of said cancellous bone region with said filler.

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 is a perspective view of a high strength osteoarticular allograft according to the instant invention;

FIG. 2 is a cross-sectional view of the high strength osteoarticular allograft of FIG. 1 along the line 2a-2a; and

FIG. 3 is a schematic illustration of a method for making a high strength osteoarticular allograft according to instant invention.

### DETAILED DESCRIPTION OF THE INVENTION

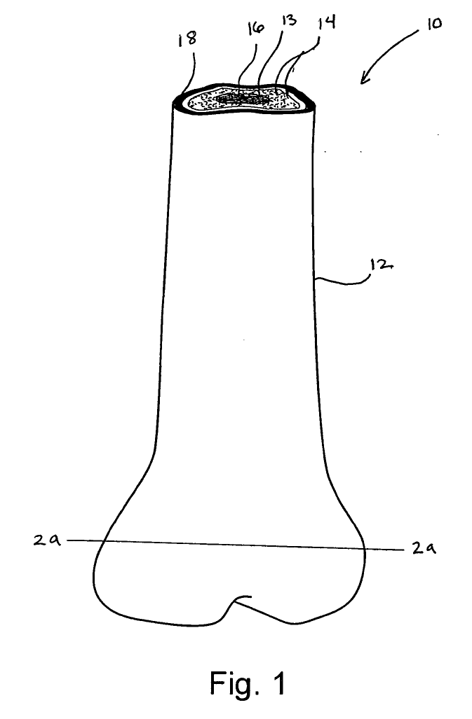

Referring to the drawings wherein like numerals indicate like elements, there is shown, in FIGS. 1 and 2, a preferred embodiment of a high strength osteoarticular allograft 10 according to instant invention. The high strength osteoarticular allograft 10 includes an osteoarticular allograft 12 having an intramedullary canal 13 and a cancellous bone region 14, and a filler 16 impregnating the intramedullary canal 13 and a portion of the cancellous bone region 14. The osteoarticular allograft 12 may further include a compact bone region 18.

The instant application, for convenience, is further discussed with regard to humans; however, the instant invention is not so limited, and it may include a high strength osteoarticular allograft for animals and a method for making a high strength osteoarticular allograft for animals.

The osteoarticular allografts 12 are generally known to persons skilled in the art. Osteoarticular allograft 12 may have any shapes; for example, they may have a shape adapted to facilitate the reconstruction after intraarticular resection for bone tumor. The osteoarticular allograft 12 may come from any source; for example, osteoarticular allograft 12 may come from another person, or a cadaver. The osteoarticular allograft 12 may have an intramedullary canal 13 and a cancellous bone region 14. The intramedullary canal 13 may have any shape; for example, it may have a hollow cylindrical shape. Intramedullary canal 13 may have any depth or any diameter; for example intramedullary canal 13 may have a depth in the range of about 1 mm to about 500 mm, and a diameter in range of about 1 mm to about 100 mm. The cancellous bone region 14, as used herein, refers to a portion of the osteoarticular allograft that consist of slender fibers and lamellae which join to form a reticular structure, resembling a lattice-work. Additionally, the osteoarticular allograft 12 may further include a compact bone region 18. The compact bone region 18, as used herein, refers to a portion of the osteoarticular allograft 12 that is dense in texture, and it is extremely porous.

Filler 16 may be any filler. For example, filler 16 may a natural material, a synthetic material, or a semi-synthetic material. Filler 16 may; for example, be a bone cement composite 16′. Bone cement composites 16′ are generally known to a person skilled in the art. Bone cement composites 16′ may be any bone cement composite; for example, the bone cement composite 16′ may be a polymer. Bone cement composite 16′ may have any viscosity; for example, bone cement composite may have a low viscosity, a medium viscosity, or a high viscosity. Viscosity of the bone cement composite 16′ is important because it effects the ability of the bone cement composite 16′ to penetrate into the cancellous bone region 14. The viscosity of a bone cement composite 16′ may, for example, be altered, i.e. decreased, by increasing the ratio of monomer to powder, chilling the monomer, and oscillatory mixing. Low viscosity bone cement composites 16′ such as low viscosity polymethylmethahacrylate bone cement are commercially available under the trademark OSTEOPAL® from Biomet, Inc. Warsaw, Ind.

The bone cement composite 16′ may further be loaded with antibiotic agents, anti-inflammatory agents, cytokines, proteins, healing factors, catalysts, combinations thereof, and the like. Antibiotic agents may be any chemical inhibiting microbial growth. For example, antibiotic agents may be selected from the group consisting of penicillin, vancomycin, gentamicin, clindamycin, combinations thereof, and the like. Cytokines may be any cytokine; for example, it may be an antigenic cytokine promoting vascularization.

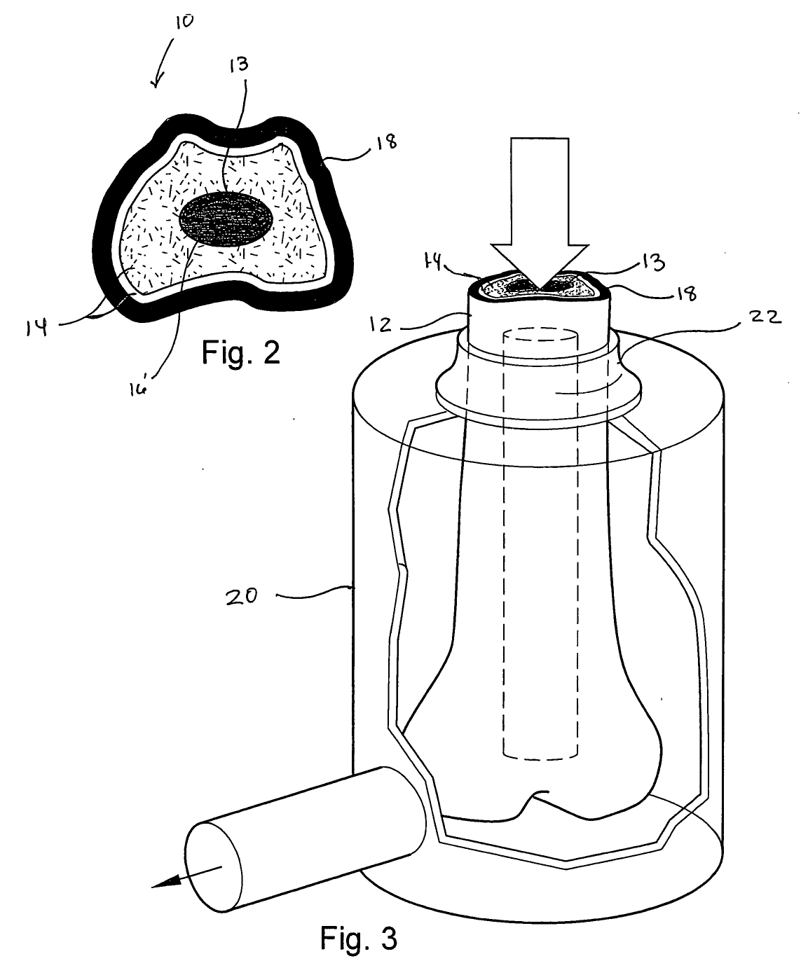

Referring to FIG. 3, the method of making a high strength osteoarticular allograft 10 employs a combination of thorough cleansing of the allograft 12, pressurization of the bone cement composite 16 while simultaneously applying vacuum around the allograft 12 to draw the bone cement composite 16′ into the cancellous bone region 14.

In operation, the osteoarticular allograft 12 is reamed with a flexible reamer (not shown); thus, having a intramedullary canal 13 with a diameter in the range of about 1 mm to about 100 mm; preferably, about 5 mm to about 20 mm. The intramedullary canal 13 may have any depth; for example, a depth in the range of 1 mm to about 500 mm. The osteoarticular allograft 12 is then thoroughly cleansed utilizing a detergent mixture (not shown). Impulse lavage technique may be used to cleanse the allograft 12. Detergent mixture (not shown) may be any detergent mixture suitable for cleansing; for example, detergent mixture may be Brij-35, Nonident P-40, Nonoxynol-3, 3% Hydrogen Peroxide, or combinations thereof. The osteoarticular allograft 12 is subsequently cleansed with a 70% Isopropyl Alcohol using impulse lavage. However, if cellular debris are not adequately removed from the osteoarticular allograft 12, the osteoarticular allograft 12 may be immersed in a detergent mixture, as described above, 70% Isopropyl Alcohol, or combinations thereof in an ultrasonic cleaner (not shown). Such ultrasonic cleaners are commercially available, for example, from Branson Ultrasonics Corporation. Furthermore, if the ultrasound fails to adequately remove the cellular debris from the osteoarticular allograft 12, then the osteoarticular allograft 12 may placed in a vacuum chamber (not shown) and perfused with a detergent mixture, as described above, 70% Isopropyl Alcohol, or combinations thereof while applying vacuum, for example, 23″ Hg, thereto. Finally, the osteoarticular allograft 12, while applying vacuum thereto, is flushed with saline solution, and then dried. Subsequently, a bone cement composite, e.g. OSTEPAL®, is prepared according to the manufacturer's instruction; for example, preparation of the bone cement composite under vacuum, room temperature, or combinations thereof. Antibiotic agents, anti-inflammatory agents, cytokines, proteins, healing factors, catalysts, combinations thereof, as described hereinabove, may be introduced into the intramedullary canal 13. Osteoarticular allograft 12 is then placed into a vacuum chamber 20, as shown in FIG. 3, and a seal 22 is formed. A cement gun (not shown) is used to inject the bone cement composite 16′ under pressure into the osteoarticular allograft 12 by, for example, a pressurizer (not shown). Simultaneously with the pressurized injection of the bone cement composite 16′ into the osteoarticular allograft 12, vacuum is applied thereto the osteoarticular allograft 12 to force the bone cement composite 16′ to permeate into the cancellous bone region 14. The movement of bone cement composite 16′ may be monitored via fluoroscopy. Once the osteoarticular allograft 12 is impregnated with the bone cement 16′, the pressure and vacuum are maintained, and the bone cement composite is allowed to be cured. In the alternative, osteoarticular allograft 12 may be perforated, for example, via drilling hole, mechanical burring, or the like, to increase filling.

The permeation of the bone cement composition 16′ into the different regions of osteoarticular allograft 12, e.g. cancellous bone region 14, and intramedullary 13 may be monitored via CT scanning. The bone cement composition 16′ may impregnate at least 30% of the cancellous bone region 14; preferably, the bone cement composition 16′ may impregnate a substantial portion of the cancellous bone region 14.

The present invention may be embodied in other forms without departing from the spirit and the essential attributes thereof, and, accordingly, reference should be made to the appended claims, rather than to the foregoing specification, as indicated the scope of the invention.

I claim:

1. A high strength osteoarticular allograft comprising:
an osteoarticular allograft having an intramedullary canal and a cancellous bone region; and
a filler impregnating both said intramedullary canal and a portion of said cancellous bone region.

2. The high strength osteoarticular allograft according to claim 1, wherein said filler impregnating at least 30% of said cancellous bone region.

3. The high strength osteoarticular allograft according to claim 1, wherein said filler impregnating a substantial portion of said cancellous bone region.

4. The high strength osteoarticular allograft according to claim 1, wherein said filler being a bone cement composite.

5. The high strength osteoarticular allograft according to claim 4, wherein said bone cement composite further including antibiotic agents, anti-inflammatory agents, cytokines, proteins, healing factors, catalysts, combinations thereof, or the like.

6. The high strength osteoarticular allograft according to claim 1, wherein said high strength osteoarticular allograft further includes antibiotic agents, anti-inflammatory agents, cytokines, proteins, healing factors, catalysts, combinations thereof, or the like.

7. A method for making a high strength osteoarticular allograft comprising the steps of:
providing a osteoarticular allograft having an intramedullary canal and a cancellous bone region;
providing a filler; and
impregnating said intramedullary canal and a portion of said cancellous bone region with said filler.

8. The method for making a high strength osteoarticular allograft according to claim 7, wherein said cancellous bone region being impregnated with said filler via exerting pressure upon said filler and simultaneously drawing vacuum on said osteoarticular allograft thereby forcing said filler substantially into said cancellous bone region.

9. The method for making a high strength osteoarticular allograft according to claim 8, wherein at least 30% of said cancellous bone region being impregnated by said filler.

10. The method for making a high strength osteoarticular allograft according to claim 8, wherein a substantial portion of said cancellous bone region being impregnated by said filler.

11. The method for making a high strength osteoarticular allograft according to claim 7, wherein said filler being a bone cement composite.

12. The method for making a high strength osteoarticular allograft according to claim 11, wherein said bone cement composite further including antibiotic agents, anti-inflammatory agents, cytokines, proteins, healing factors, catalysts, combinations thereof, or the like.

13. The method for making a high strength osteoarticular allograft according to claim 7, wherein said high strength osteoarticular allograft further includes antibiotic agents, anti-inflammatory agents, cytokines, proteins, healing factors, catalysts, combinations thereof, or the like.
